(19) United States
(12) Patent Application Publication
(10) Pub. No.: US 2007/0031477 A1
(43) Pub. Date: Feb. 8, 2007

(54) Methods and devices for holding a composition

(75) Inventors: Spiros Fotinos, Kolonaki, Athens (GR); Ligia-Stephania Panaitescu, Attiki (GR)

Correspondence Address: BROMBERG & SUNSTEIN LLP, 125 SUMMER STREET, BOSTON, MA, 02110-1618, US

(73) Assignee: LAVIPHARM S.A., Peania, Attica (GR)

(21) Appl. No.: 10/550,991

(22) PCT Filed: Mar. 26, 2004
(86) PCT No.: PCT/US04/09044

§ 371 Date: Jul. 13, 2006

Related U.S. Application Data
(60) Provisional application No. 60/457,752, filed on Mar. 26, 2003.

(51) Int. Cl. A61K 9/70

(52) U.S. Cl. 424/443

(57) ABSTRACT

Methods and devices for holding a composition are described. A first embodiment includes a support substrate that supports an array of film segments having an active agent-containing composition, the film segments being attached to the support substrate by a pattern of adhesive. Another embodiment involves a support substrate supporting film segments having the active agent-containing composition, the embodiment including a sealing material that is heat sealed to the support substrate to contain the film segments. A third embodiment involves a substrate holding the film segments in blister cavities; a sealing material is attached to the substrate to contain the segments in the blister cavities. Other embodiments refer to cold sealing support substrates and sealing material, methods of delivering a composition without separating a film segment from a substrate backing, and other alterations to the sealing material.

### TECHNICAL FIELD

The present invention relates to techniques and devices for packaging, or holding, compositions, and in particular, compositions containing an active agent.

### BACKGROUND ART

In manufacturing pharmaceuticals, cosmetics, food supplements as well as other products to induce some type of effect, an important consideration is how the quantities, or specific doses, of a composition are distributed to the consumer. Techniques of manufacturing the composition should be integrated with methods and devices for holding and/or packaging the composition; this may allow advantages such as streamlining production labor and decreased manufacturing costs. In the particular instance where the composition is embodied with an active agent-containing film, the film may be relatively sensitive, fragile, and easily deformable or damaged. Thus, integrating manufacturing techniques with design considerations for packaging and holding compositions may have the added advantages enhancing product stability and quality. Simultaneously, the risk of product deterioration may be decreased by minimizing exposure of the product to the environment (e.g. light, air, heat, moisture, etc.), isolating the product from contact with people, and facilitating product handling (e.g. preventing the stacking of films that may adhere to one another).

### SUMMARY OF THE INVENTION

One embodiment of the invention is directed toward a device for holding an active agent-containing composition. The device includes a support substrate; a pattern of adhesive in contact with one side of the support substrate; and an array of discrete film segments removably attached to the support substrate by contact with the adhesive, each film segment including the active agent-containing composition. The side of the support substrate facing the film segments, the array of film segments, and the pattern of adhesive may be sterile. The adhesive may be substantially inert to the film segments, and the pattern of adhesive may be parallel lines. The device may also be stored in a separate container. The support substrate used in embodiments of the invention may be transparent. Each discrete film segment may contain a uniform quantity of the active agent.

Another embodiment of the invention is directed toward a device for holding an active agent-containing composition that includes a support substrate; an array of discrete film segments removably attached to one side of the support substrate, each film segment including the active agent; and a sealing material, the material covering the array of discrete film segments, a surface of the material heat sealed to the one side of the support substrate at locations surrounding at least one discrete film segment. Elements of the embodiment may be sterile. The surface of the sealing material may be heat sealed to the one side of the support substrate at locations surrounding each discrete film segment. The sealing material may be transparent or tear resistant. A portion of the surface of the sealing material may include a non stick coating to reduce adhesion between the film segments and the sealing material. The array of discrete film segments may be a dried polymer gel. Support substrates may be made of paper or a plastic sheet, and may be transparent. One side of the support substrate may include a coat of polyethylene that is optionally glossy or unexposed to Corona treatment. The sealing material may include a polyester sheet double-side coated with polyethylene (which may be ultra-dimensional stable), the surface of the polyester sheet heat sealed to the a side of the support substrate that is glossy. The corners of the support substrate may be detached from the sealing material.

In a related embodiment of the invention, the device for holding a composition includes a sealing material covering the array of discrete dosage units, a surface of the sealing material reattachably adhering to the one side of the support substrate by a pressure sensitive adhesive. The discrete dosage units may be an array of discrete film segments. A surface of the sealing material may be reattachably adhered to the one side of the support substrate such that at least one discrete dosage unit is surrounded by a reattachably adhered contact area between the sealing material and the support substrate.

In another embodiment of the invention, a device for holding a composition includes a support substrate; an array of dosage units contacting one side of the support substrate, each dosage unit including the composition; and a surface of a sealing material covering the array of dosage units. At least one selected portion of the surface of the sealing material includes a non stick coating where the sealing material contacts the dosage units. The non stick coating may include either a fluorochemical or silicon-based compound. The surface of the sealing material may be attached to the support substrate, the non stick coating not being present at a location where the sealing material is attached to the support substrate. The array of dosage units may be an array of discrete film segments attached to the support substrate. The non stick coating on a surface of the sealing material may be on a plurality of locations corresponding to locations where the sealing material contacts each dosage unit, or may be on one continuous area. Optionally, the array of dosage units are attached to the support substrate by interposed adhesive.

Some embodiments of the invention may be directed toward a device for holding an active agent-containing composition that includes a support substrate; and a dried solution layer removably attached to the support substrate, the dried solution layer having the active agent-containing composition. The dried solution layer may be configured to deliver the active agent-containing composition to a patient by direct contact of the dried solution layer with the patient. The dried solution layer may also include a colorant to visually distinguish the dried solution layer from the support substrate.

Other embodiments of the invention refer to methods of creating the devices in previously described embodiments of the invention.

In another embodiment of the invention, a method for holding an active agent-containing composition includes providing a substrate; forming blister cavities in the substrate; covering the substrate with a film including the active agent-containing composition; displacing film segments into each blister cavity; removing the film unassociated with the segments; superposing a sealing material, the sealing material covering the segments of film and in contact with the substrate; and attaching the sealing material at a plurality of locations where the sealing material and substrate are in contact. The formation of the plurality of blister cavities and the displacing the film may occur substantially simultaneously. The displacing of film segments may include punching the film to displace the film segments into the blister cavities. The sealing material may be attached to the substrate using heat sealing, or by applying pressure at locations where a pressure sensitive adhesive is sandwiched between the sealing material and the substrate. The sealing material may be lacquered aluminum foil. Each segment of film may include a uniform quantity of the active agent.

An alternate method of holding an active agent-containing composition, in accord with an embodiment of the invention, includes providing a substrate with a plurality of blister cavities; providing quantities of the active agent, each quantity contained in one of the plurality of blister cavities; superposing a sealing material, the material covering the quantities of the active agent-containing composition and in contact with the substrate; and attaching the sealing material at a plurality of locations where the sealing material and substrate are in contact. The sealing material may be attached to the substrate using heat sealing, or by applying pressure at locations where a pressure sensitive adhesive is sandwiched between the sealing material and the substrate.

In another embodiment of the invention, a method of applying a composition in a dosage unit to a dermal surface of a patient includes providing a dosage unit having the composition on a side of a final substrate that is larger in area than the dosage unit; holding the final substrate without touching the dosage unit; bringing the final substrate toward the dermal surface to bring the dosage unit into contact with the dermal surface; and pressing against the final substrate to apply the composition to the dermal surface. The method may also include one or more of moistening the dermal surface before the step of bringing the final substrate toward the dermal surface, and rubbing the final substrate to facilitate application of the composition to the dermal surface. When the dosage unit is a film segment, providing the dosage unit may include providing a dried solution forming a film on an intermediate substrate; removing the film from the intermediate substrate; forming a film segment from the film; and attaching the film segment to the final substrate. Providing the dosage unit may also include providing a substrate having a plurality of dosage units, and separating the substrate into a plurality of final substrates, each final substrate having at least one dosage unit. Alternatively, providing the dosage unit may include providing a plurality of final substrates, each final substrate having at least one dosage unit, each of the plurality of final substrates attached to at least one other final substrate. Optionally, providing the plurality of final substrates may include providing a substrate having a plurality of dosage units attached thereto; and perforating the substrate to form the plurality of final substrates.

In another embodiment of the invention, a method for holding an active agent-containing composition includes intermittently applying a solution containing the active agent to a substrate; and drying the intermittently applied solution to form film segments. Applying the solution may include at least one of spraying, printing, and casting the solution intermittently. When printing is utilized, the printing may be performed on a fabric, or by a gavure-type process. The film segments may be configured to deliver the active agent-containing composition to a patient by directly contacting the film segments with the patient.

Various embodiments of the invention that include an active agent-containing composition may be utilized in an application that includes at least one of therapeutics, cosmetics, food supplements, wound healings, herbals, botanicals, aromatherapy, veterinary applications, agrochemicals, and cleansing applications.

### BRIEF DESCRIPTION OF THE DRAWINGS

The foregoing features of the invention will be more readily understood by reference to the following detailed description, taken with reference to the accompanying drawings, in which:

FIGS. 1A and 1B are a plan view and side view, respectively, of a device for holding active agent-containing compositions, in accordance with an embodiment of the invention, whereby film segments are attached onto a substrate by means of a pattern of adhesive.

FIGS. 2A-2C are a series of side views illustrating the steps for making a device for holding film segments to a substrate material without the use of an adhesive, covered by a sealing material, embodiment.

FIGS. 3A-3C is a series of schematic side views of the steps for making a device for holding active agent-containing compositions in a blister cavity-formed substrate attached to a sealing material embodiment.

FIG. 4A depicts a side view of dosage units that are sandwiched between a support substrate and a sealing material that are attached by a cold seal process around the edges of the support substrate and sealing material, in accordance with an embodiment of the invention.

FIG. 4B depicts a side view of dosage units that are sandwiched between a support substrate and a sealing material that are attached by a cold seal process at locations around each dosage unit, in accordance with an embodiment of the invention.

FIG. 4C depicts a top view of dosage units that are surrounded by a pattern of adhesive that do not attach the corners of a support substrate, according to an embodiment of the invention.

FIG. 4D depicts a top view of dosage units that are sandwiched between a support substrate and a sealing material, the sealing material irreversibly attached to an edge of the support substrate to form a reattachable flap, in accord with an embodiment of the invention.

FIGS. 5A and 5B present top views of embodiments of the invention that utilize a subdivided substrate with perforations, each subdivision containing a film segment and attached to the other subdivisions.

FIGS. 6A and 6B present top views of embodiments of the invention that utilize a non-stick coating applied to a sealing material to reduce adhesion with dosage units.

### DETAILED DESCRIPTION OF THE SPECIFIC EMBODIMENTS

The embodiments described herein refer to methods and devices for holding compositions, including compositions containing active agents. The methods and devices may be employed to hold a plurality of dosage units, such as, for example, film segments, having an active agent-containing composition. The film segments may be formed from a film that includes the composition and may be configured in any desired shape and size.

For some embodiments described herein, film segments 115, 215, 315, 415, 515 may be composed from a film of dried polymer solution or dried polymer gel, either of which may also be hydrophilic in nature before drying occurs. The polymer gel or polymer solution may be formulated with a level of viscosity sufficient to allow the gel or solution to hold a specific configuration while drying occurs. The drying time depends upon a variety of factors that may include the type of solvents used, their concentration, the nature and concentration of the other constituents, the film thickness, the sensitivity of active components to high temperature, the air mass convected over the gel or solution, and the speed of film formation. For example, with heat sensitive materials, relatively low temperatures are preferred, requiring longer drying periods. Some embodiments may require a drying time of several minutes.

Polymers that may be utilized in the film segments 115, 215, 315, 415, 515 and the film 110, 210, 310, include, for example, polyurethane, polyvinyl pyrrolidone (PVP) and hydroxypropyl methylcellulose (HPMC). A variety of materials including cellulose, starch and their derivatives, proteins, gelatins, etc may also be used. Other polymers include polymers of natural origin. One such embodiment is a plant prolamine, an example being gliadin, combined with other additives of natural origin; such additives may include a polar lipid, such as a ceramide. Each film segment 115, 215, 315, 415, 515 contains some amount of the composition to be delivered. A solvent, preferably ethanol when PVP or HPMC is used, is added to the polymer to make the solution.

The composition utilized in a dosage unit may belong to any category (e.g., compositions that are absorbed or applied to a patient by direct contact of a dosage unit with a patient). Some embodiments are preferably drawn to compositions with one or more active agents. An active agent may have a therapeutic effect (e.g., a pharmaceutical agent) or may have activity in other applications such as cosmetics, food supplements, wound healings, herbals, botanicals, aromatherapy, veterinary applications, agrochemicals (e.g., pesticides), cleansing applications (e.g., disinfectants), and other agents, in general. The active agent may be directed toward oral or topical application, such as epidermal, transdermal, or transmucosal (e.g., respiratory tract, oral cavity, vagina, penile surface, etc). The amount of active agent may be the same in each dosage unit and may also constitute a particular dosage of the active agent. Beyond active agents, compositions may also include thickeners, preservatives, colorants, microencapsulants, and other components that may alter the delivery or nature of an active agent.

Embodiments of the dried polymer film segments have thicknesses of 0.03, 0.05, and 0.06 millimeters, each segment having a surface area varying between 0.8 and 3.0 square centimeters. The film may also contain additional components that alter particular physical and chemical properties. For example, a film may include a plasticizer and an antioxidant.

In a specific example, dried polymer film segments include an active agent for treating erectile dysfunction, a polymer, a plasticizer, and an antioxidant. The dried film segments are circularly shaped with a surface area of 2.0 cm² and a thickness of 0.05 mm.

Though some embodiments described herein refer specifically to holding film segments with an active agent, these embodiments may also be used to hold other carriers with the active agent including various types of transdermal, epidermal, or transmucosal delivery systems (e.g. patches, tablets, capsules, and pills).

In some instances, sterilization of the device holding a composition helps preserve the composition in a particular state before their use. Thus alternatives to the embodiments described herein include application of methods and techniques known in the art to sterilize compositions, film segments, substrates, adhesives, sealing materials, and other materials. Examples of suitable sterilization techniques include gamma-irradiation and electron beam exposure.

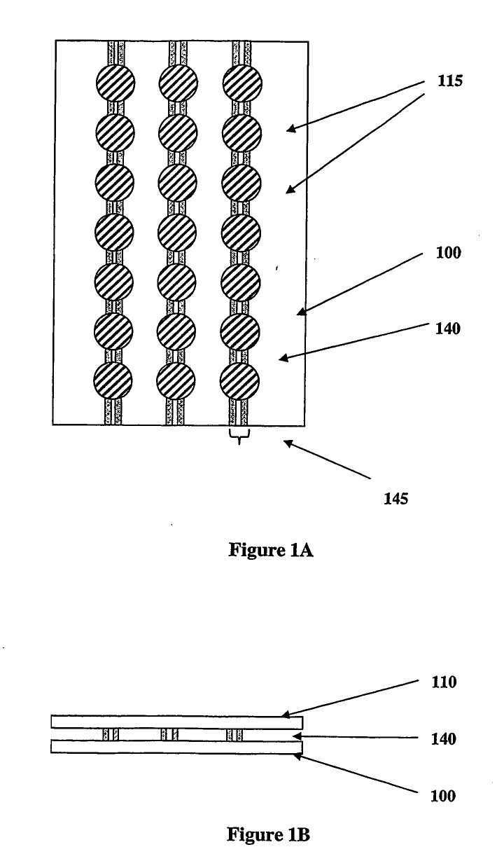

Referring to FIG. 1, a support substrate 100 is shown holding a plurality of film segments 115 each having an amount of the active agent. The film segments 115 are removably attached to the support substrate 100 by an adhesive 140, allowing the film segments 115 to be separated from the support substrate 100 by a user without damaging the segments 115.

The support substrate 100 may be composed of any type of material that allows the film segments 115 to be removably attached to the support substrate 100 with an appropriate adhesive 140. Preferred substrate materials include polyester, polystyrene, high density polyethylene, substrates treated with or made with a fluorochemical (e.g., Teflon®), paper, super-calendered (smoothed) paper, and polyurethane films. Laminates of the aformentioned materials may also be utilized. The support substrate 100 may be a release liner for the film segments 115. In addition, the support substrate 100 may be transparent, allowing a person to view the film segments 115 through the support substrate 100.

Selection of the adhesive is based in part upon the compatibility of the adhesive with one or more components of the formulation. Inertness of the adhesive is generally preferred. For example, salicylic acid containing formulations have been shown to interact chemically and are, therefore, incompatible with adhesives containing free hydroxyl and/or carboxyl groups. Combinations of salicylic acid and adhesives with free hydroxyl and/or carboxyl groups may suffer from one or more of stability problems, deformation of the formulation or adhesive layer; the problems may lead to the deterioration of the product's properties (e.g., activity and physical appearance). Thus, these combinations, and contact with such, are preferably avoided. With regard to tacking properties, the adhesive may impart a peeling load/width within a range of 5-50 gf/inch, as measured by a 180° peel test, to attach the film segments to the support substrate, while allowing the segments to be removed from the substrate without damage. Examples of possible adhesives for use with the embodiment include, but are not limited to, Duro-Tak® (produced by National Starch), Gelva® (produced by Solutia), and other commercially available polyacrylic adhesives.

The adhesive 140 may be applied in a particular pattern on the support substrate 100. For example, the adhesive 140 may constitute sets of closely spaced parallel lines 145 that allow the film segments 115 to be arranged in the pattern of a rectangular matrix, as depicted in FIG. 1A. In a particular embodiment, the adhesive is applied in three pairs of adhesive lines, each line being approximately 80 millimeters long, 2 millimeters wide, and 0.02 millimeters thick. The adhesive 140 allows the film segments 115 to be removably attached to the support substrate 100. In addition, the adhesive 140 is chosen such that it does not adversely affect the chemical or physical nature of the active agent or film segments 115.

Referring to FIGS. 1A and 1B, one method for making the device, in accord with embodiments of the invention, includes the steps of: (a) providing a final support substrate 100 with a pattern 145 of adhesive 140 in contact with a side of the support substrate 100; (b) removably attaching a film 110 with the active agent to the final support substrate 100 through contact with the pattern of adhesive 145 as shown in FIG. 1B; and (c) segmenting the film 110 to form an array of film segments 115 attached to the final support substrate 100 through contact with the adhesive 140. In a particular embodiment of the method, the step of removably attaching the film 110 to the final support substrate 100 includes providing the film 110 with the active agent removably attached to an initial support substrate; bringing the film 110 in contact with the pattern of adhesive 140; and delaminating the initial support substrate from the film 110, leaving the film 110 attached to the final support substrate 100.

In the particular method described above, the initial support substrate may be composed of any material that has the property of adhering to the film 110 more weakly than the final support substrate 100 adheres to the film 110. The film's adherence to the initial support substrate may be an intrinsic property of the two surfaces, or may be facilitated by the use of an adhesive. Possible initial support substrates include release liners. Release liners for the initial support substrate may include a side, which contacts the film 110, being treated with either a silicon-based compound or a fluorochemical such as Teflon®. For example, in the case of a silicon-based compound, a polyethylene terephthalate substrate is coated with a mixture containing a polysiloxane with vinyl-functional side chains or hydroxyl-functional side chains; a crosslinker (e.g., a polysiloxane with Si—H functionality); a platinum or tin complex as a catalyst; and optionally solvent and/or additives. The mixture is heated to react the components to form a network of polysiloxanes. Any remaining solvent is subsequently removed. The resulting coating is a thin silicone-like layer.

Segmenting the film 110 into an array of film segments 115 may include one or more processes, non-exhaustive examples being cutting and removing of the film 110. In a particular segmenting process, kiss-cutting of the film 110 is performed. Kiss-cutting involves making cuts that penetrate the film 110, while not penetrating the support substrate 100. The kiss-cutting is followed by removal of a portion of the film from the support substrate 100, leaving discrete film segments 115 that adhere to the support substrate 100 by contact with adhesive 140, as depicted in FIG. 1A. The remaining film that is not utilized as film segments 115 may be discarded or recycled to formulate more film.

The steps of the method may be performed to produce a continuous product. In such an instance, an additional step of cutting the final support substrate 100 with attached film segments 115 into discrete sets is included. Machinery, such as the Allied Gear and Machine Co.'s Flexomaster IB press, may be utilized to perform one or more steps of the method continuously. In an embodiment of the method, the step of segmenting the film may be performed by the Flexomaster IB press. In this embodiment, the film 110, support substrate 100, and adhesive 140 are provided as a continuous laminate that is rolled up. The laminate is unrolled by the machine and kiss-cut by a first cutting tool, and perforated and scored by a second cutting tool. The machine may also be configured to perform some preprocessing steps on the laminate, such as printing, before kiss cutting occurs. The cutting tools are configured to accommodate the specific film thickness and support substrate utilized. Film 110 that is not utilized in the film segments, scrap, is delaminated from the original laminate and wound up in a roll. The remaining laminate, including film segments 115 attached to a support substrate 100 through contact with adhesive 140, is rewound on a roll for further processing.

One or more sets of the film segments 115 attached to the final support substrate 100 may be packaged in a pouch, or other container, to protect the assembly from potential hazards, that may include physical damage, contamination, light exposure, or other environmental elements.

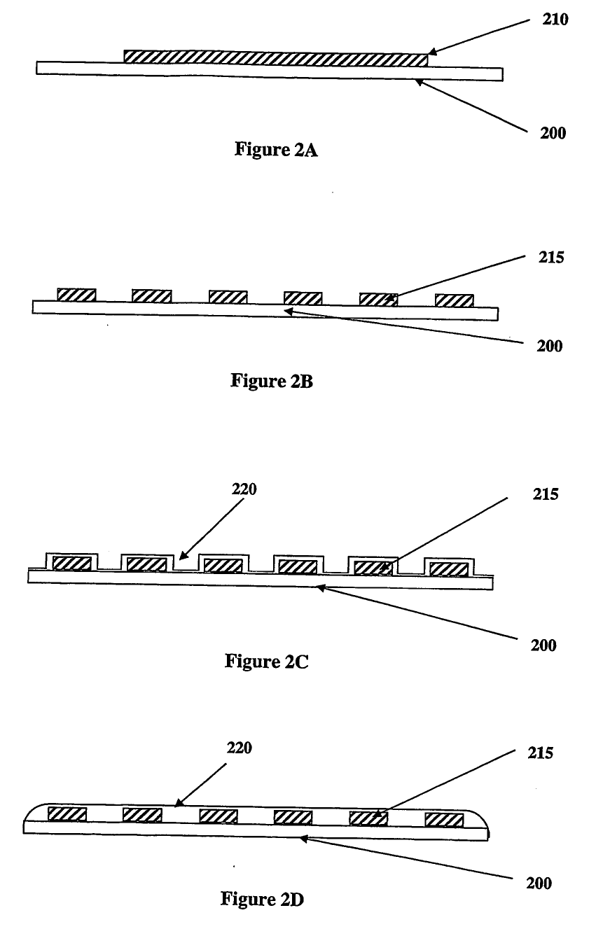

Another embodiment of a device for holding an active agent-containing composition is shown in FIG. 2C. In this embodiment, film segments 215 are removably attached to a support substrate 200 without the use of an adhesive; adhesion of the film segments 215 to the support substrate 200 is due to the nature of the interaction between the segments 215 and the support substrate 200. Minor self-adhesive properties of the film segments, substrate surface roughness (surface irregularities created by a particular treatment method), and temporarily induced electrostatic charges provide some examples of mechanisms that result in adhesion between the segments 215 and support substrate 200. A sealing material 220 covers the film segments 215 so that the segments are protected in a sandwich between the support substrate 200 and the sealing material 220. The sealing material 220 is heat sealed to the support substrate 200, to enclose the film segments in a sealed package. The film segments 215 are protected from potential hazards that may include physical damage, contamination, light exposure, and other environmental conditions.

The support substrate 200 may be composed of any type of material that allows the film segments 215 to be removably attached to the support substrate 200 and does not cause adverse physical or chemical changes to the nature of the active agent or film segments 215. Examples of acceptable support substrate materials include paper or plastic sheets. The support substrate 200 may be a release liner. In a particular embodiment, the support substrate 200 is composed of a polyethylene-coated paper. The side of the polyethylene-coated paper that adheres to the film segments 215 is glossy, and not Corona treated, i.e. the side has a smooth, reflective surface that has not been treated to introduce any surface irregularities. This insures that adhesion of the film segments 215 to the support substrate 200 is not so strong as to prevent the segments 215 from being removed without damage. The support substrate 200 may also be made of a transparent material, allowing persons to view the film segments through the support substrate 200.

One embodiment of a sealing material 220 utilizes a polyester sheet double side coated with polyethylene, the surface of the film that contacts the support substrate 200 being glossy. In particular, this embodiment of the sealing material 220 may be configured to be especially stable. Examples of the embodiment include the ultra-dimensional stable polyester films produced by Loparex. The films are polyester films coated with a layer of low-density polyethylene on each side. These films do not lose moisture when heated, and, therefore, do not change their dimensions upon heating, i.e., they do not tend to curl, and are thus considered very stable liners.

The sealing material 220 may be composed of any material that may be heat sealed to the support substrate 200. The sealing material 220 may be transparent, allowing the film segments 215 to be seen through the sealing material 220. The sealing material 220 may also be chosen to be tear resistant.

The sealing material 220 may be heat sealed to the support substrate 200 such that each film segment 215 is isolated from the other film segments 215 as shown in FIG. 2C. The sealing material may also be intentionally unattached to the support substrate in particular locations, e.g. the corners of the sealing material, to allow ease of separation of the sealing material from the support substrate by a user.

As shown in FIGS. 2A-2D, a method for malting the device includes the steps of applying a film 210 having the active agent to a side of a support substrate 200;

segmenting the film 210 into film segments 215; covering the film segments 215 with a sealing material 220; and heat sealing the sealing material 220 to the support substrate 200. The film segments 215 are attached to the support substrate 200, but may be removed therefrom without damage to the film segments 215. In particular, the method may include using a polymer gel or polymer solution to make the film 210, and include the step of drying the film 210 after the film 210 is applied to a support substrate 200.

The step of segmenting the film 210 may include kiss-cutting the film 210 and removing the film portions that are not utilized as film segments 215 on the support substrate 200. Excess film may either be discarded or recycled to formulate more film. A continuous process may be employed to carry out the steps of this method; in such a case, the method further includes a step of cutting the attached support substrate 200 and sealing material 220 holding a plurality of film segments 215 to create sets of the held film segments 215.

In a preferred embodiment of the method, a coating machine applies the polymer solution or polymer gel to the support substrate, forming a laminate sheet. The solution or gel is dried, and the laminate wound up into a roll. The Allied Gear and Machine Co.'s Flexomaster IB press, that was described earlier to create the embodiment in FIG. 1, can be utilized with the roll created by the coating machine to kiss-cut and delaminate the scrap material not being packaged.

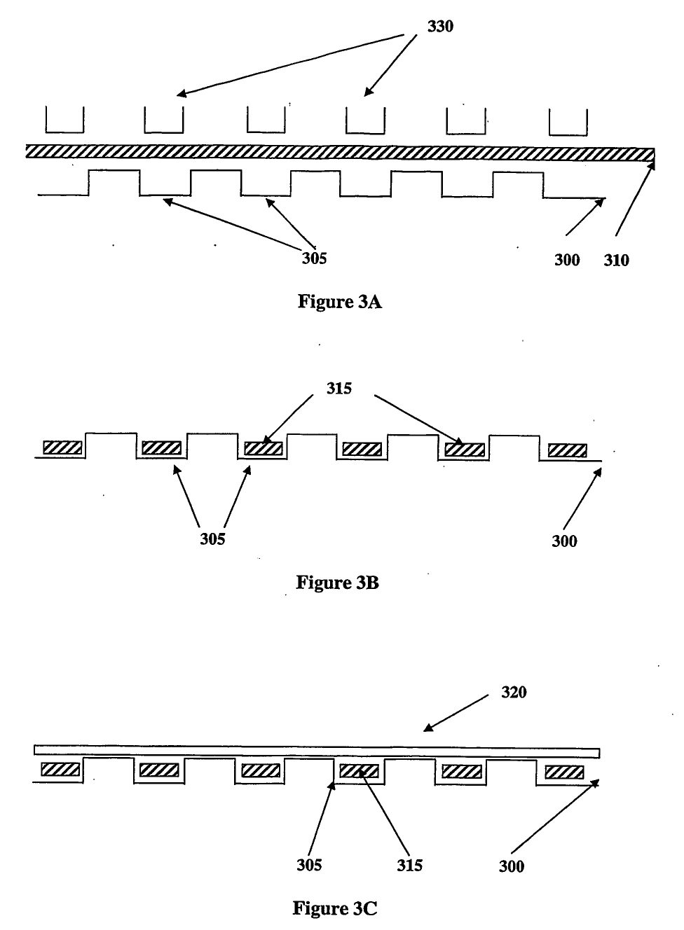

Referring to FIGS. 3A-3C, an alternate method of producing a device for holding film segments with an active agent-containing composition is described. Discrete film segments 315 including the active agent are deposited into individual blister cavities 305 of a substrate material 300. The film segments 315 may be formed from an original film 310 containing the active agent. A sealing material 320 is placed over the blister cavities 305 containing the film segments 315 and attached to the substrate material 300 to contain the film segments 315. The film segments 315 may be free from attachment while contained within the blister cavity 305 covered by the sealing material 320.

The substrate material 300 may be any material to which blister cavities 305 may be formed. Blister cavities 305 are stable impressions in the substrate material 300 that are sized to contain individual film segments 315, as depicted in FIGS. 3B and 3C. When the substrate 300 is oriented such that the blister cavities 305 are in a concave downward orientation, as depicted in FIGS. 3A-3C, individual film segments 315 may be contained within each blister cavity 305, with an open top surface as depicted in FIG. 3B. Examples of suitable materials for the substrate material 300 include, but are not limited to, polyvinyl chloride, a combination of polyvinyl chloride and polyvinylidene chloride, and aluminum.

The formation of blister cavities 305 in the substrate material 300 and depositing of film segments 315 into the blister cavities 305 may be performed using various techniques. In one technique, film segments 315 are provided on a blister cavity-free substrate material. The film segments 315 may be formed and provided on the substrate material 300 utilizing the methods described earlier to form the embodiments depicted by FIG. 2. The film segments 315 may be held in place on the substrate material 300 by electrostatic charges. Next, a machine forms blister cavities 305 in the substrate material 300 at positions where film segments 315 are located. The blister cavities 305 may be formed by any technique known to those in the art. In one embodiment, the blister cavities 305 are formed by moving the substrate 300 over an appropriately sized and shaped orifice in a surface of a machine. The substrate material 300 is drawn into the orifice, deforming permanently into a shape confined by the orifice and creating the blister cavity 305. The process of deforming the substrate material 300 may utilize heat or mechanical force. For example, a vacuum may be used to provide suction to a surface of the substrate to deform the substrate locally to form a blister cavity. In another example, a punch may be used to contact the substrate material surface and deform it locally into a blister cavity. Optionally, the portion of the substrate to be deformed may be heated before deformation takes place to facilitate blister cavity formation. Since each blister cavity 305 is formed adjacent to a film segment 315, upon cavity formation the segment 315 is simultaneously deposited into the cavity 305.

In another technique, a film 310 is placed over a blister cavity-free substrate material. A machine forms blister cavities 305 in the substrate material while also displacing a film segment 315 from the film 310 into each blister cavity 305. In one embodiment, the blister cavities 305 are formed by drawing the substrate material 300 into an orifice, as discussed above. At approximately the same time, film segments 305 are displaced from the film 310 by impinging a punch 330 against the film 310 on a side opposite the substrate material 300. The shape of the punch may be chosen to create a desired film segment size. The punch 330 may have a surface that impinges upon the film 310 that is flat or curved. The punch contact surface may also have the shape of an annular ring or frame of any desired shape or size. The punch 330 preferably has edges that are sharp enough to cut the film 310. The punch 330 forms film segments 315 that are deposited into the blister cavities 305. The punch 330 is subsequently withdrawn. Alternatively, one or more punches may be used to preform blister cavities in a substrate, followed by the punching of a film disposed over the substrate to deposit segments into the blister cavities.

In a third technique, a substrate material 300 with preformed blister cavities 305 is provided. The blister cavities 305 may be formed by any technique known in the art including the particular technique described above. A film 310 with the active agent is positioned over the substrate material 300 on a side of the substrate material 300 in which the blister cavities 305 protrude away from the film 310, as depicted in FIG. 3A. A machine impinges the film 310 with a punch 330 on a side opposite the substrate material 300 in positions over the blister cavities 305. The punch contact creates a film segment 315 which is separated from the film 310 and deposited into the blister cavity 305.

Subsequent to the depositing of film segments into the blister cavities, a sealing material is used to contain the film segments within the cavities. The sealing material 320 may be any material that may be attached to the substrate material 300. In a particular embodiment, the sealing material 320 is heat sealable to the substrate material 300. One example of a sealing material 320 is lacquered aluminum foil. Other properties of the sealing material 320 that may be desirable include transparency and tear resistance.

The sealing material 320 is placed over openings of the blister cavities 305 that contain the film segments 315. Portions of the sealing material 320 are in contact with the substrate material 300. In a particular configuration, the sealing material 320 contacts the portions of the substrate 300 that do not constitute a blister cavity 305. Locations of contact between the sealing material 320 and substrate material 300 may be heat sealed, thus containing the film segments 315 within the blister cavities 305. The heat sealing may take place around each blister cavity to ensure isolation of each film segment. The corners of the substrate or sealing material may not be heat sealed to allow easier separation of the layers when access to the film segments is desired by a consumer.

The method may be practiced in a continuous process that forms a continuous substrate with blister cavities 305, film segments 315, and sealing material 320. A plurality of blister cavities 305 andlor film segments 315 may be formed in a batch process that is carried out successively. The continuous method would include the step of cutting the substrate 300 to provide discrete sets of the film segments 315.

In an alternative to this blister cavity packaging method, the film segments are preformed and deposited into preformed blister cavities in a substrate material. In this embodiment, the method and device are not necessarily limited to the packaging of film segments but may include packaging of active agents in other forms including patches, tablets, and pills.

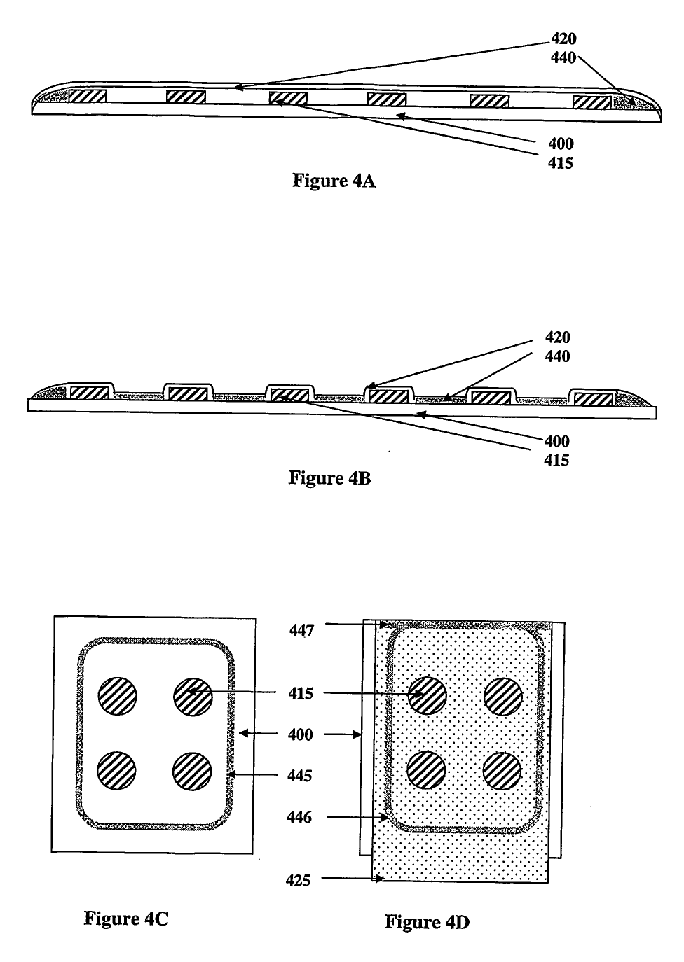

Dosage units 415 may be held between a support substrate 400 and a sealing material 420, the support substrate 400 attached to the sealing material 420 by a cold seal process, in an embodiment of the invention as depicted in FIGS. 4A-4D. The cold seal process utilizes a pressure sensitive adhesive 440, 445, 446 that reattachably adheres the sealing material 420 to the support substrate 400, i.e., the sealing material 420 may be separated from the support substrate 400 and subsequently reattached by applying pressure between the two layers 400, 420 at the positions where adhesive 440, 445, 446 is applied. The adhesive 440, 445, 446 may be applied to either layer 400, 420 or both. The adhesive 440, 445, 446 may also have the property of only adhering to itself or a surface with a particular chemical nature. In a particular embodiment, the substrate is composed of a multilaminate of paper/polymer/foil/polymer, such as Type 7340 (Phoenix Health Care Products, LLC, Milwaukee, Wis.), and the adhesive is a compounded natural rubber latex, such as PHX 3/03AX; PHC 3/03AX, or CSC 3/03AX (Phoenix Health Care Products, LLC, Milwaukee, Wis.).

As shown in FIGS. 4A and 4B, the pattern of applied adhesive may surround one or more dosage units, and may be in any pattern that is advantageous. As shown in FIG. 4C for example, the adhesive pattern 445 is directed away from the corners of the support substrate 400 to facilitate separation of the support substrate 400 and sealing. material 420. Alternatively, a portion of the support substrate and sealing material may be permanently bonded, while another portion of the support substrate and sealing material are reattachably adhered. For example, as depicted in FIG. 4D, the support substrate 400 and sealing material 420 are permanently bonded 447 at an edge to form a reattachable flap 425 with the sealing material, the adhesive pattern 446 serving to reattachably adhere the flap 425 to the support substrate 400.

Use of a cold seal process may provide several advantages. A consumer may peel the sealing material from the support substrate to obtain one of the dosage units, and subsequently reattach the layers to keep other dosage units protected until use. Thus cold sealing may alleviate the costs associated with packaging individualized dosages. A cold-seal process may also avoid the risk of heat damage that may be sustained to a substrate, sealing material, or dosage unit if a heat sealing process is utilized, as well as need for specialized machinery.

The use of a cold seal process may be utilized with several of the aforementioned embodiments of the invention including when dosages are attached to the substrate, either by adhesive or self-adhesive nature of a dried film segment, or placed in a blister cavity configuration. Dosage units may be film segments, as described herein, or other carriers such as mini-tablets and suppositories containing active agents. Use of a cold seal process may also allow the use of radiation to sterilize embodiments of the invention without affecting the particular properties of the embodiment (e.g., adhesive bonding, active agents' potency, substrate material's optical or mechanical properties, sealing material's optical or mechanical properties, and others).

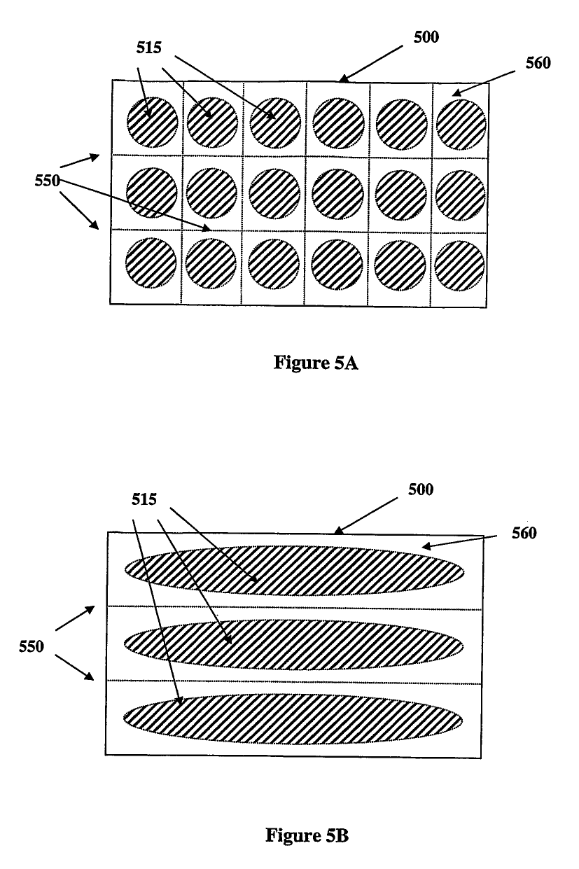

Film segments, carrying an active agent and attached to a support substrate, may be used to apply the active agent to a dermal surface of a patient in another embodiment of the invention. As shown in FIGS. 5A and 5B, a substrate 500 with film segments 515 distributed thereon may be divided into subdivisions. Each subdivision 560 may be attached to one film segment 515, the side of the subdivision contacting the film segment having a surface area greater than the film segment. One film segment may hold a particular dosage of active agent. A film segment may then be placed in contact with a dermal surface to transfer the contacting agent. In particular, a patient may manipulate the film segment to contact a dermal surface by handling the subdivision and not contacting the film segment. Subsequently, upon transfer of the active agent, the subdivision may be disposed, again without contact with the agent by handling the subdivision only. Thus patient exposure to the contacting agent may be limited to only the dermal contact desired.

A film segment may include the use of a colorant that facilitates the visual distinction between a film segment and the support substrate or subdivision that the film segment to which the film segment is attached. Thus successful transfer of the film segment to a dermal surface may be more easily identified by the substantial absence of colorant on the support substrate or subdivision.

Agent transfer may be facilitated by applying pressure to the film through the subdivision. In addition or optionally, the dermal surface may be moistened with saliva, water, or some other liquid to promote agent transport. Transfer of the agent may include transfer of part, or the entire, film segment that contacts the dermal area.

Formation of the film segments that are attached to a support substrate may be performed in numerous manners. In one example, a solution containing the contacting agent may be applied to an intermediate support substrate. The applied solution is dried to form a film. The dried film may be removed as a sheet from the intermediate support substrate, and separated into film segments (e.g., cutting the sheet). The film segments are attached to the support substrate (e.g., polyethylene or polypropylene) that is subsequently subdivided. This method can advantageously invoke use of most, if not all, of the film.

In another example, a solution containing the active agent-containing composition may be applied in an intermittent manner to the substrate that is subsequently subdivided. The intermittently applied solution is dried to form the film segments. Intermittent solution application may be by any known conventional method including solution casting, spraying, or printing (e.g., “silk-screen printing” or “gravure-type”). In the specific instance where silk-screen printing is utilized, types of substrates that may be utilized include non-woven material, fabrics, and polyesters that are optionally siliconised. The intermittent pattern of applied solution, and the amount of solution applied for forming each film segment, may vary as required for a particular application. For example, the segments may be shaped to conform to a particular application location such as the lips or eyes. FIGS. 5A and 5B show two possible layouts of solution application. Forming film segments in this manner saves resources by eliminating wasted film and the use of an intermediate substrate.

Dividing a substrate into a plurality of subdivisions may be performed in a number of manners. In one example, the substrate is cut into separate subdivisions that may be subsequently packaged. In another example, the substrate may be perforated 550 into subdivisions, the subdivisions still connected with each other as depicted in FIGS. 5A and 5B. The perforations allow a patient to subsequently separate the subdivisions for individual use. In a third example, a patient may simply tear, without preset guidance, the substrate to remove a film segment attached to the torn subdivision.

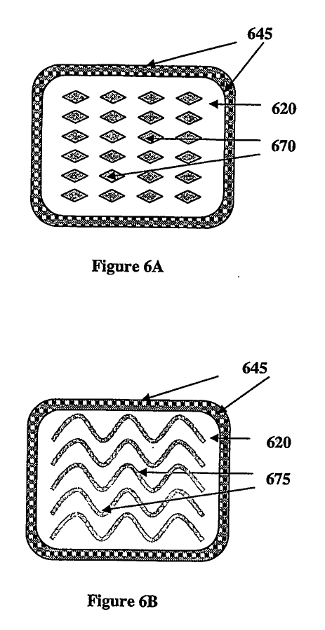

In embodiments of the invention that utilize a sealing material, the sealing material may have a selected portion of its surface that limits adhesion of the sealing material with the dosage units that may contact the sealing material. As shown in an embodiment of the invention shown in FIG. 6A, a surface of a sealing material 620 in contact with a plurality of dosage units associated with a substrate is treated at a plurality of locations 670 with a non-stick coating. When the sealing material 620 is attached to a substrate at location 645, the locations 670 correspond with areas where dosage units may contact the sealing material 620. The coating 670 reduces the adhesion of the dosage units with the sealing material 620, to help prevent the dosage units from being damaged when the sealing material 620 and substrate are separated. Such an embodiment may be especially useful in situation where “cold flow” of adhesive, which attaches a dosage unit to a desired site, may cause undesirable adhesion between the substrate and sealing material at position close to a dosage unit, and/or adhesion between the dosage unit and the substrate and/or the sealing material, possibly damaging the dosage unit when the substrate and sealing material are separated.

Non-stick coatings that may be utilized include silicon-based coatings and fluorochemical-based coatings (e.g., Teflon®). Such coatings may be applied to the surface of the sealing material in any pattern (e.g., lines, islands, spots, etc.) that may be advantageous to preventing damage to the dosage units when the sealing material is separated from the substrate. In FIG. 6A, the non-stick coating is applied at a plurality of discrete locations 670 corresponding to the contact points between the sealing material 620 with the dosage units. Alternatively, as shown in FIG. 6B, the non-stick coating is applied to a large area 675 of the sealing material 620 surface that includes potential contact locations between the dosage units and sealing material 620. In general, the non-stick coatings are applied to the sealing material in a manner to avoid contact with locations of the sealing material that are intended to bind the sealing material to the substrate (e.g., the edges 645 of the sealing material 620 shown in FIGS. 6A and 6B).

The aforementioned embodiments are intended to be merely exemplary; numerous variations and modifications will be apparent to those skilled in the art, including variations and modification that utilize one or more particular features from one or more various described embodiments. All such variations and modifications are intended to be within the scope of the present invention.

1. A device for holding an active agent-containing composition, the device comprising:
a support substrate;
a pattern of adhesive in contact with one side of the support substrate; and an array of discrete film segments removably attached to the support substrate by contact with the pattern of adhesive, each film segment including the active agent-containing composition.

2. A device according to claim 1, wherein the one side of the support substrate, the array of discrete film segments, and the pattern of adhesive are sterile.

3. A device according to claim 1, wherein the pattern of adhesive comprises parallel lines.

4. A device according to claim 1, wherein the adhesive is substantially inert to the film segments.

5. A device according to claim 1, the device further comprising a container for holding the support substrate, the pattern of adhesive, and the array of discrete film segments.

6. A device according to claim 1, wherein the support substrate is transparent.

7. A device according to claim 1, wherein each discrete film segment includes a uniform quantity of an active agent.

8. A device according to claim 1, wherein the active agent-containing composition is utilized in an application that includes at least one of therapeutics, cosmetics, food supplements, wound healings, herbals, botanicals, aromatherapy, veterinary applications, agrochemicals, and cleansing applications.

9-50. (canceled)

51. A method of holding an active agent-containing composition, the method comprising:
providing a final support substrate, one side of the substrate in contact with a pattern of adhesive;
removably attaching a film including the active agent-containing composition to the final support substrate, the film in contact with the pattern of adhesive; and
segmenting the film attached to the final support substrate into an array of discrete film segments attached to the final support substrate.

52. A method according to claim 51, the method further comprising sterilizing the array of film segments, the pattern of adhesive, and the one side of the final support substrate.

53. A method according to claim 51, wherein the pattern of adhesive comprises parallel lines.

54. A method according to claim 51, the method further comprising enclosing the final support substrate, the pattern of adhesive, and the array of discrete film segments in a container.

55. A method according to claim 5l, wherein removably attaching the film includes
contacting a film removably attached to an initial support substrate with the pattern of adhesive, the initial support substrate located on a side of the film opposite the pattern of adhesive; and
delaminating the initial support substrate from the film.

56. A method according to claim 55, wherein a side of the initial support substrate in contact with the film is treated with a fluorochemical.

57. A method according to claim 55, wherein a side of the initial support substrate in contact with the film is coated with silicon-based compound.

58. A method according to claim 51, wherein segmenting the film includes removing portions of the film from the final support substrate.

59. A method according to claim 51, wherein the steps are performed by a continuous process.

60. A method according to claim 51, wherein each discrete film segment includes a uniform quantity of an active agent.

61. A method according to claim 51 further comprising:
superposing a sealing material over the array of discrete film segments attached to the final support substrate, the sealing material including a non-stick coating on at least a portion of the surface of the sealing material, wherein the non-stick coating reduces adhesion between the surface of the sealing material and at least one film segment when the sealing material contacts the at least one film segment.

62. A method according to claim 51, wherein the active agent-containing composition is utilized in an application that includes at least one of therapeutics, cosmetics, food supplements, wound healings, herbals, botanicals, aromatherapy, veterinary applications, agrochemicals, and cleansing applications.

63-117. (canceled)
